(19) United States
(12) Patent Application Publication
(10) Pub. No.: US 2009/0177114 A1
(43) Pub. Date: Jul. 9, 2009

(54) Echogenic needle aspiration device

(76) Inventors: Yem Chin, Burlington, MA (US); Adam Cohen, Arlington, MA (US); Michal Weisman, Allston, MA (US); Robert DeVries, Northborough, MA (US); Oscar Carrillo, JR., Attleboro, MA (US)

Correspondence Address: KRIEGSMAN & KRIEGSMAN, 30 TURNPIKE ROAD, SUITE 9, SOUTHBOROUGH, MA, 01772, US

(21) Appl. No.: 12/316,663

(22) Filed: Dec. 15, 2008

Related U.S. Application Data
(60) Provisional application No. 61/007,811, filed on Dec. 13, 2007.

(51) Int. Cl. A61B 10/02

(52) U.S. Cl. 600/565

(57) ABSTRACT

An echogenic needle aspiration device. In one embodiment, the device includes an echogenic needle, the needle including a tubular sidewall having a non-circular transverse cross-section over at least a portion of its length. The non-circular cross-sectional shape of the needle may be provided, for example, by radially spacing one or more straight, longitudinally-extending fins, ribs or other projections about the periphery of the needle and/or by providing the needle with a polygonal cross-sectional shape.

### CROSS-REFERENCE TO RELATED APPLICATIONS

The present application claims the benefit under 35 U.S.C. 119(e) of U.S. Provisional Patent Application Ser. No. 61/007,811, filed Dec. 13, 2007, the disclosure of which is incorporated herein by reference.

### BACKGROUND OF THE INVENTION

The present invention relates generally to needle aspiration devices and relates more particularly to echogenic needle aspiration devices.

Early detection and diagnosis are prerequisites for proper treatment of many medical conditions. For example, in the case of cancer, early detection and diagnosis are often critical to the success of the patient's treatment and recovery. In many instances, such detection and diagnosis require one to remove a tissue sample from a patient so that the tissue sample may be subjected to a suitable form of examination. These steps of removal and examination are often referred to in the art, both collectively and individually, as biopsy. Biopsy tissue samples may be obtained from a patient by various techniques. One of the longer-standing techniques for obtaining a biopsy sample is an open, surgical technique that involves making a conventional surgical incision in the patient in the vicinity of a tissue of interest and then excising one or more samples from the tissue of interest. Unfortunately, this tissue-sampling technique is very invasive, is expensive to perform, and requires a considerable recovery time.

A more recent alternative to the above-described surgical technique is an endoscopic technique employing a needle aspiration device (also commonly referred to in the art as a fine needle aspiration device). A needle aspiration device is a device that typically includes a hollow needle, an inner tube, and an outer tube. The hollow needle is coaxially mounted within the distal end of the inner tube, the proximal end of the inner tube being adapted for connection to a vacuum source, such as a syringe. The hollow needle and the inner tube are slidably mounted within the outer tube. In this manner, when the device is intended for sampling, the needle and the inner tube may be positioned relative to the outer tube so that the needle extends distally beyond the distal end of the outer tube and, thus, is adapted to be inserted into a tissue. By contrast, when the device is not intended for sampling, the needle and the inner tube are typically positioned relative to the outer tube so that the needle is sheltered within the outer tube. In a typical endoscopic technique employing a needle aspiration device, the distal end of an endoscope is introduced into a patient, typically through the mouth of the patient. The distal end of the needle aspiration device is then passed through a channel of the endoscope and is positioned near the tissue of interest. Next, the needle of the needle aspiration device is advanced distally beyond the distal end of the outer tube, under ultrasound the needle is inserted into the tissue of interest (typically being moved back and forth to promote the shearing of cells from the tissue), and suction is applied to the needle through the inner tube. Due to the insertion of the needle into the tissue and the application of suction, a tissue sample typically becomes lodged within the needle. The needle is then retracted into the outer tube, the needle aspiration device is withdrawn from the patient, and positive pressure from the suction device is applied to expel the tissue sample from the needle onto/into a suitable examination medium.

As can be appreciated, when using an endoscopically-introduced needle aspiration device to obtain a tissue sample, it is highly desirable that the physician be able to visualize the insertion of the needle into the targeted tissue. In some cases, such as where the targeted tissue is located within the gastrointestinal tract, such visualization may be accomplished by direct visualization using an optical channel of the endoscope. However, in certain other cases, the targeted tissue is not located directly within the gastrointestinal tract; consequently, the insertion of the needle into the targeted tissue cannot be visualized by direct visualization. An example of such a case is where the targeted tissue is located within the pancreas. In such a case, the pancreas is typically accessed by advancing the distal end of the endoscope into the stomach and then inserting the needle of the needle aspiration device through the stomach wall and into the pancreas. As can be appreciated, because the distal end of the endoscope and the needle are located on opposite sides of the stomach wall, the insertion of the needle into the pancreas cannot be directly visualized using an optical channel of the endoscope. For this reason, other visualization techniques have typically been used in these types of endoscopic procedures. One such alternate visualization technique is ultrasonography. For endoscopic procedures of the type described above, ultrasonography typically involves the use of an ultrasonic transceiver or probe positioned at the distal end of the endoscope. The ultrasonic probe emits sound waves and then collects the return sound waves, which are then used to form an image of the object that caused the reflection of the sound waves. By adjusting the frequency of the sound waves emitted from the probe, images of the object at different distances from the probe may be obtained.

Although ultrasonography has been used to obtain images of medical devices inside a patient's body that would otherwise not be possible through direct visualization, the image quality is often less than optimal. This is, in part, because many medical devices do not inherently possess optimal reflective properties with respect to ultrasound waves. To remedy this, certain medical devices have been designed to possess enhanced sound wave reflectivity, i.e., echogenicity. For example, in the case of needle aspiration devices, it is known to roughen, by bead-blasting, the outer surface of the needle to improve its echogenicity.

Also, in U.S. Pat. No. 4,401,124, inventors Guess et al., which issued Aug. 30, 1983, and which is incorporated herein by reference, there is disclosed a reflection enhancement of a biopsy needle for use in conjunction with an apparatus for pulse-echo ultrasound imaging of an area of the body. According to this patent, the reflection enhancement is in the form of a helical groove that is provided on the exterior surface of the tip of the biopsy needle.

Additionally, in PCT International Publication Number WO 2006/044374 A1, which was published Apr. 27, 2006, and which is incorporated herein by reference, there is disclosed an echogenic medical device and a method of forming an echogenic surface. In particular, the aforementioned publication discloses medical devices that are made visible under ultrasonic or magnetic imaging techniques by adding a series of depressions or voids to their surfaces. The depressions or voids are desirably placed at more than one angle to the surface in order to enhance the visibility of the surface. Laser-machining can be used to make depressions or voids that are symmetric with respect to the surface and another series of depressions or voids that are non-symmetric. The pattern of voids is also varied by using more than one side of void, the depth of the voids, and the distribution of voids, i.e., more voids in some areas than others.

In addition, in U.S. Pat. No. 6,053,870, inventor Fulton, III, which issued Apr. 25, 2000, and which is incorporated herein by reference, there is disclosed an ultrasonic visible surgical needle. This patent discloses, in one embodiment, a surgical needle manufactured of stainless steel or some other material which provides an interface with human tissue that results in an ultrasonic echo. One or more transverse notches are cut into the sidewall of the needle. In one tested embodiment, the transverse notch intersects the circumference of the needle over approximately 100°. Thus the notch cuts through to the lumen. The notch is a wedge-shaped notch defining first and second oblique walls in the sidewall of the lumen. Where these walls are planar, the plane of each wall intersects the axis of the needle at an acute angle. The acute angle may range from 15° to 75° and in a preferred embodiment are each 45°. The walls created by the notch provide a large amount of reflective surface area.

Additionally, in U.S. Pat. No. 6,358,211 B1, inventor Mamayek, which issued Mar. 19, 2002, and which is incorporated herein by reference, there is disclosed an ultrasound lucent apparatus and methods for use in a wide range of invasive imaging and surgical procedures. In one exemplary embodiment of this patent, an apparatus is disclosed that comprises an elongate wire body having an outer surface and a longitudinal axis. The wire body includes a plurality of corner reflectors disposed on an embossed portion of the outer surface. The wire body is adapted to be inserted into a body lumen or a patient vasculature. In this manner, use of corner reflector technology enhances the ultrasound reflective nature to increase the acoustical reflectivity of the wire body.

Other documents of interest include U.S. Pat. No. 5,843,023, inventor Cecchi, which issued Dec. 1, 1998; U.S. Pat. No. 6,454,702, inventor Smith, which issued Sep. 24, 2002; U.S. Pat. No. 6,723,052 B2, inventor Mills, which issued Apr. 20, 2004; and U.S. Patent Application Publication No. US 2004/0106891 A1, inventors Langan et al., which was published Jun. 3, 2004, all of which are incorporated herein by reference.

### SUMMARY OF THE INVENTION

It is an object of the present invention to provide a novel echogenic needle aspiration device.

Therefore, according to one aspect of the invention, there is provided an echogenic needle aspiration device, the echogenic needle aspiration device comprising (a) a first catheter, the first catheter having a proximal end, a distal end and a lumen, the lumen of the first catheter being adapted to receive suction; and (b) a needle, the needle comprising a proximal end, a distal end and a tubular sidewall, the tubular sidewall defining a lumen and having a length, the proximal end of the needle being fixedly coupled to the distal end of the first catheter, with the lumina of the first catheter and the needle in fluid communication with one another, the tubular sidewall comprising a non-circular transverse cross-section over at least a portion of the length of the tubular sidewall. The non-circular transverse cross-sectional shape may be provided, for example, by radially spacing one or more fins or other projections about at least some of the periphery (exterior) of the portion or by providing at least some of the portion with a polygonal cross-sectional shape.

The present invention is also directed to a novel echogenic medical device. According to one aspect, the echogenic medical device comprises (a) a needle, the needle comprising a proximal end, a distal end and a lumen; and (b) a stylet, the stylet being slidably received in the lumen of the needle, the stylet including an echogenic distal end. The echogenic distal end of the stylet may be shaped to include one or more depressions and/or one or more protuberances. More specifically, the echogenic distal end of the stylet may include a length having a non-circular transverse cross-section. The non-circular transverse cross-section may be provided by radially spacing one or more fins or other projections about at least some of the periphery (exterior) of the distal end or by providing at least some of the distal end with a polygonal cross-sectional shape.

The present invention is additionally directed at a needle visible to ultrasound, the needle comprising a tubular sidewall, the tubular sidewall defining a lumen and having a length, the tubular sidewall having a non-circular transverse cross-section over at least a portion of the length of the tubular sidewall. The tubular sidewall may have a non-circular transverse cross-section over substantially the entirety of the length of the tubular sidewall or over only one or more portions of the length of the tubular sidewall. In the latter case, the tubular sidewall may include one or more spaced-apart portions of non-circular transverse cross-section, the spaced apart-portions either having identical or different non-circular transverse cross-sections.

For purposes of the present specification and claims, various relational terms like “top,” “bottom,” “proximal,” “distal,” “upper,” “lower,” “front,” and “rear” are used to describe the present invention when said invention is positioned in or viewed from a given orientation. It is to be understood that, by altering the orientation of the invention, certain relational terms may need to be adjusted accordingly.

Additional objects, as well as features and advantages, of the present invention will be set forth in part in the description which follows, and in part will be obvious from the description or may be learned by practice of the invention. In the description, reference is made to the accompanying drawings which form a part thereof and in which is shown by way of illustration various embodiments for practicing the invention. The embodiments will be described in sufficient detail to enable those skilled in the art to practice the invention, and it is to be understood that other embodiments may be utilized and that structural changes may be made without departing from the scope of the invention. The following detailed description is, therefore, not to be taken in a limiting sense, and the scope of the present invention is best defined by the appended claims.

### BRIEF DESCRIPTION OF THE DRAWINGS

The accompanying drawings, which are hereby incorporated into and constitute a part of this specification, illustrate various embodiments of the invention and, together with the description, serve to explain the principles of the invention. In the drawings wherein like reference numerals represent like parts:

FIG. 1 is a fragmentary, side view of a first embodiment of an echogenic needle aspiration device constructed according to the teachings of the present invention, the echogenic needle aspiration device being shown with its needle in an extended position;

FIG. 2 is a fragmentary, side view of the echogenic needle aspiration device of FIG. 1, the echogenic needle aspiration device being shown with its needle in a retracted position;

FIG. 3 is a fragmentary, longitudinal section view of the echogenic needle aspiration device of FIG. 1, the echogenic needle aspiration device being shown with its needle in an extended position;

FIG. 4 is an enlarged transverse section view of the echogenic needle aspiration device of FIG. 1 taken along line 1-1;

FIGS. 5(a) through 5(e) are enlarged transverse section views of alternate needles for use in the echogenic needle aspiration device of FIG. 1;

FIG. 6 is a side view of a further alternate needle for use in the echogenic needle aspiration device of FIG. 1;

FIGS. 7(a) and 7(b) are enlarged transverse section views of the needle of FIG. 6 taken along lines 2-2 and 3-3, respectively;

FIG. 8 is a fragmentary, side view of a second embodiment of an echogenic needle aspiration device constructed according to the teachings of the present invention, the echogenic needle aspiration device being shown with its needle and its stylet in an extended position; and

FIGS. 9(a) and 9(b) are distal end and fragmentary side views, respectively, of an alternate stylet for use in the echogenic needle aspiration device of FIG. 8.

### DETAILED DESCRIPTION OF PREFERRED EMBODIMENTS

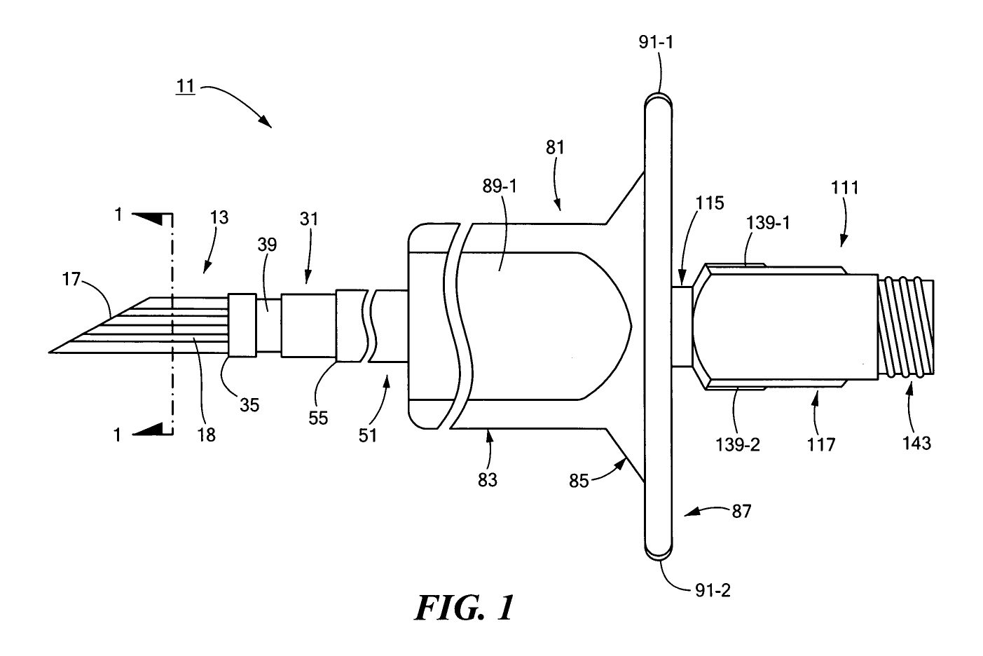

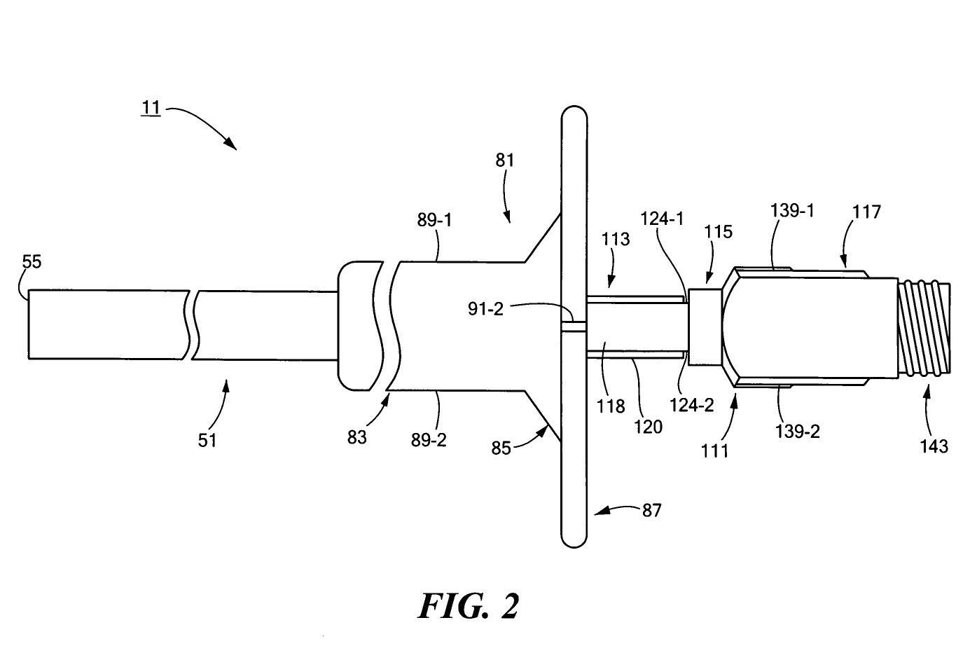

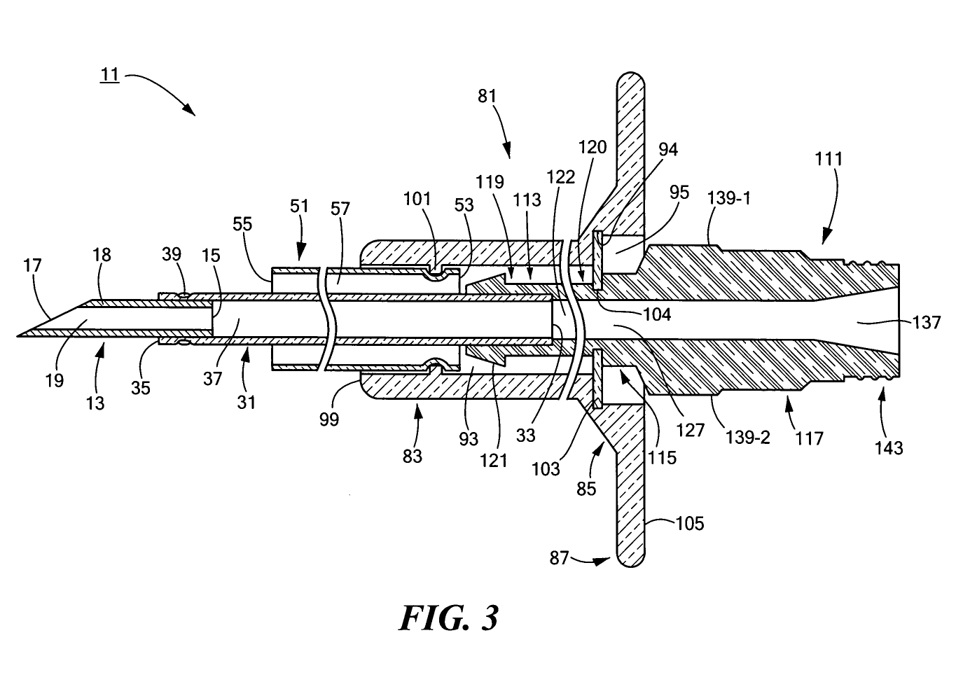

Referring now to FIGS. 1 through 3, there are shown various views of a first embodiment of an echogenic needle aspiration device constructed according to the teachings of the present invention, said echogenic needle aspiration device being represented generally by reference numeral 11.

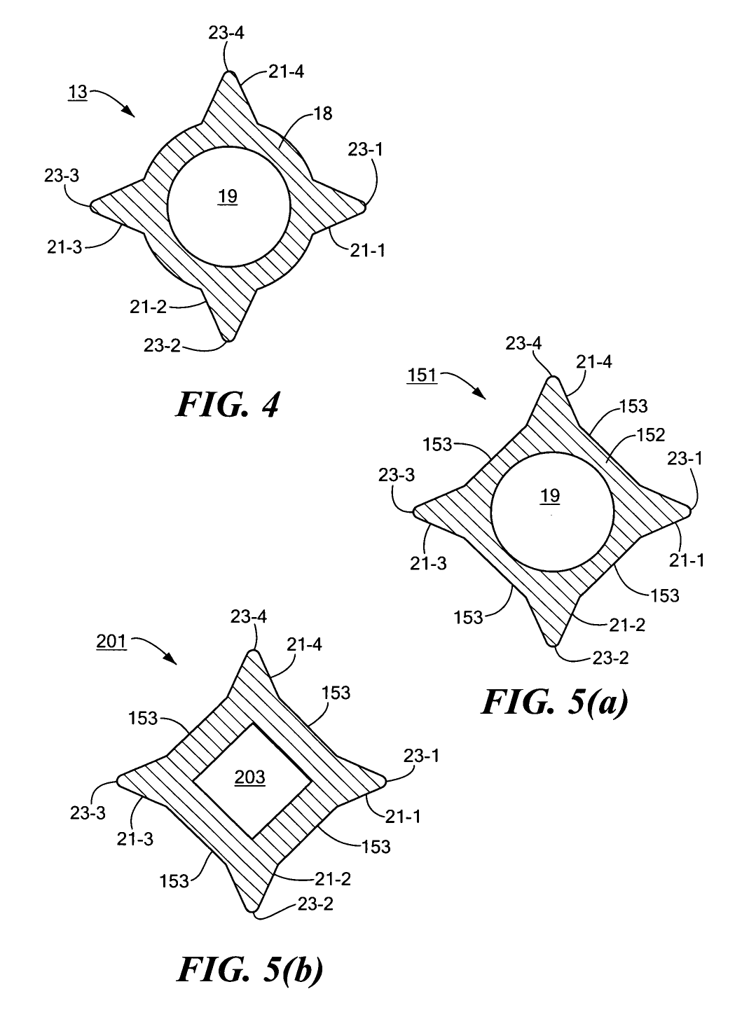

Device 11 may comprise a hollow needle 13 (needle 13 also being shown separately in FIG. 4). Needle 13, in turn, may include an elongated, tubular member shaped to include a blunt proximal end 15, a distal end 17 forming a sharp tip, a side wall 18 and a longitudinal lumen 19. Needle 13 may be made of a metal, a metal alloy, or any other material readily observable by ultrasound. In order to enhance its echogenicity, needle 13 may have a non-circular, transverse cross-sectional shape. For example, in the present embodiment (seen best in FIG. 4), side wall 18 may include a plurality of straight, longitudinal ribs 21-1 through 21-4 evenly spaced about its circumference and extending radially outwardly from lumen 19. As viewed from an end of needle, 13, ribs 21-1 through 21-4 are generally triangular in shape and have rounded vertices 23-1 through 23-4, respectively. (Although ribs 21-1 through 21-4 are evenly spaced over the entire circumference of needle 13, it should be understood that needle 13 may also be provided with a non-circular, transverse cross-sectional shape using only one rib or using a plurality of ribs positioned over only a portion of the circumference of needle 13.)

Needle 13 may be a unitary structure made, for example, by one or more of the following processes: machining, acid-etching, photo-etching, laser-cutting, coining, extruding, rotary swaging, and plating. Alternatively, instead of being a unitary structure, needle 13 may include a composite of structures joined together or added one to another. For example, needle 13 may be hard-coated using a nickel-plating process or coated with a diamond-like coating, thereby increasing the hardness of the metal surface to make it more echogenic. Alternatively, needle 13 may be made by joining a non-circular surface to a circular needle.

Device 11 may also comprise an inner catheter 31. In the present embodiment, inner catheter 31 may include an elongated, unitary, flexible member made of, for example, a suitable medical grade plastic. Inner catheter 31 may be shaped to include a proximal end 33, a distal end 35 and a longitudinal lumen 37. Proximal end 15 of needle 13 may be disposed within lumen 37 of inner catheter 31 and may be securely retained therewithin by a tubular band 39 crimped around the outside of catheter 31 against needle 13, with distal end 17 of needle 13 extending distally a short distance from distal end 35 of catheter 31.

Device 11 may further comprise an outer catheter 51. In the present embodiment, outer catheter 51 may include an elongated, unitary, flexible member made of, for example, a suitable medical grade plastic. Outer catheter 51 may be shaped to include a proximal end 53, a distal end 55 and a longitudinal lumen 57. Outer catheter 51 may be appropriately dimensioned to slidably receive inner catheter 31 and needle 13 coaxially within lumen 57. In this manner, needle 13 may be alternately extended distally from outer catheter 51, as when taking a tissue sample, and retracted into outer catheter 51, as when not in use.

Device 11 may further comprise an outer hub 81. In the present embodiment, outer hub 81 comprises an elongated, unitary, tubular, rigid member made of, for example, a suitable medical grade plastic. Outer hub 81 may be shaped to include a distal stem portion 83, an intermediate shoulder portion 85, and a proximal collar portion 87. Stem portion 83 may have an outer profile that is generally cylindrical and further may comprise a pair of opposing flattened surfaces 89-1 and 89-2 that extend longitudinally. Shoulder portion 85 may have an outer profile that is generally conical, tapering outwardly from stem portion 83 to collar portion 87. Collar portion 87, which may be generally cylindrical in outer profile, may be shaped to include embossed indicia 91-1 and 91-2, the purpose of which will be discussed further below. A longitudinal lumen may be provided in outer hub 81, said longitudinal lumen comprising a first portion 93, a second portion 94, and a third portion 95. First portion 93 may extend proximally from distal end 99 of hub 81 to second portion 94. An internal flange 101 provided in stem portion 83 extends into first portion 93 at an intermediate point located a short distance proximal to distal end 99. Flange 101 may be appropriately dimensioned so that proximal end 53 of catheter 51, which is freely received in first portion 93, may be securely retained within outer hub 81. If desired, outer hub 81 may be insert-molded around proximal end 53 of outer catheter 51, with internal flange 101 being sized to frictionally engage catheter 51 in a highly retentive manner. Second portion 94, which may be located within shoulder portion 85 and which may extend between first portion 93 and third portion 95, may be greater in diameter than each of first portion 93 and third portion 95. A washer 103 may be fixedly mounted within second portion 94, washer 103 having a generally oval aperture 104, the purpose of which will be described below. If desired, outer hub 81 may be insert-molded around washer 103. Third portion 95, which may be smaller in diameter than second portion 94 but may be greater in diameter than first portion 93, may extend proximally from second portion 94 to proximal end 105 of hub 81.

Device 11 may further comprise an inner hub 111. In the present embodiment, inner hub 111 may include an elongated, unitary, tubular, rigid member made of, for example, a suitable medical grade plastic. Inner hub 111 may be shaped to include a distal stem portion 113, an intermediate neck portion 115 and a proximal body portion 117. Stem portion 113, which may be generally cylindrical in outer profile, except for a pair of opposing flattened surfaces 118 (only one such surface 118 being shown) that may extend longitudinally, may be shaped to include a slotted distal section 119 and a tubular proximal section 120. Distal section 119 may have a bifurcated barb 121 at its distal end. Proximal end 33 of inner catheter 31 may be fixedly mounted within slotted distal section 119 of stem portion 113 by a friction fit. (If desired, slotted distal section 119 may be provided with serrations to help grip inner catheter 31.) Tubular proximal section 120 may be shaped to include a longitudinal lumen 122 and a pair of proximal notches 124-1 and 124-2 along its outer surface. Stem portion 113 may be partially inserted into outer hub 81, with barb 121 being appropriately sized relative to aperture 104 of washer 103 so that barb 121 may be inserted through aperture 104 during assembly of injection needle 13 but, thereafter, cannot easily be withdrawn proximally through aperture 104. In addition, tubular proximal section 120 may be dimensioned relative to aperture 104 of washer 103 so that, when stem portion 113 and aperture 104 are properly aligned rotationally, proximal section 120 may be moved back and forth through aperture 104 and so that, when stem portion 113 is fully inserted into outer hub 81 (with notches 124-1 and 124-2 disposed within aperture 104), stem portion 113 may be rotated 90 degrees relative to aperture 104, thereby preventing proximal section 120 from being moved translationally relative to outer hub 81.

Neck portion 115, which may be generally cylindrical in outer profile, may be shaped to include a longitudinal lumen 127, lumen 127 being aligned with lumen 122 of proximal section 120. Neck portion 115 may be appropriately dimensioned to serve as a stop to limit insertion of inner hub 111 into outer hub 81.

Proximal body portion 117, which may be generally rectangular in outer profile, may be shaped to include a lumen 137, lumen 137 being aligned with lumen 127 of neck portion 115. Embossed indicia 139-1 and 139-2 may be provided on opposing surfaces of proximal body portion 117, indicia 139-1 and 139-2 being provided to be alignable with indicia 91-1 and 91-2, respectively, to indicate the rotational alignment of inner hub 111 to outer hub 81, such as when one wishes to prevent longitudinal movement of inner hub 111 relative to outer hub 81. The proximal end of proximal body portion 117 may be shaped to include an externally threaded connector 143 adapted for use with a needle-less syringe or a similar source of suction.

Device 11 may be used in a comparable fashion to conventional needle aspiration devices.

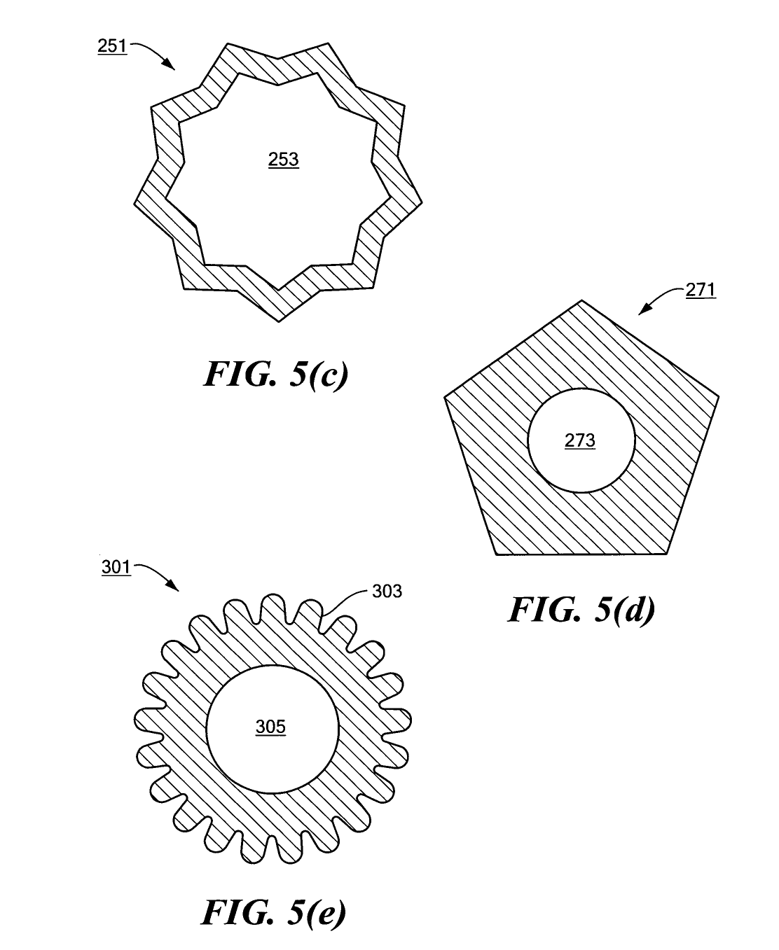

Referring now to FIGS. 5(a) through 5(e), there are shown enlarged transverse section views of alternate needles for use in echogenic needle aspiration device 11. More specifically, in FIG. 5(a), a needle 151 is shown, needle 151 differing from needle 13 in that the sidewall 152 of needle 151 may include flat portions 153, instead of rounded portions, extending peripherally between ribs 21-1 through 21-4. In FIG. 5(b), a needle 201 is shown, needle 201 differing from needle 151 in that needle 201 may have a square-shaped lumen 203. In FIG. 5(c), a needle 251 is shown, needle 251 differing from needle 13 in that needle 251 may have a nonagonal star-like cross-sectional shape and a nonagonal lumen 253. In FIG. 5(d), a needle 271 is shown, needle 271 differing from needle 13 in that needle 271 may have a pentagonal cross-sectional shape and a circular lumen 273. In FIG. 5(e), a needle 301 is shown, needle 301 differing from needle 13 in that needle 301 may have a gear-shaped exterior surface 303 and a circular lumen 305.

It should be understood that the various needle embodiments disclosed above are merely illustrative and that the needle of the present invention may assume any non-circular cross-sectional shape (with the lumen of the needle being either circular or any non-circular shape). The non-circular cross-sectional shape of the present needle may be provided to the needle by positioning one or more straight, longitudinal fins, ribs or other projections about a portion or the entirety of the periphery (exterior) of the needle and/or by providing the needle with a polygonal cross-sectional shape over at least a portion of its circumference.

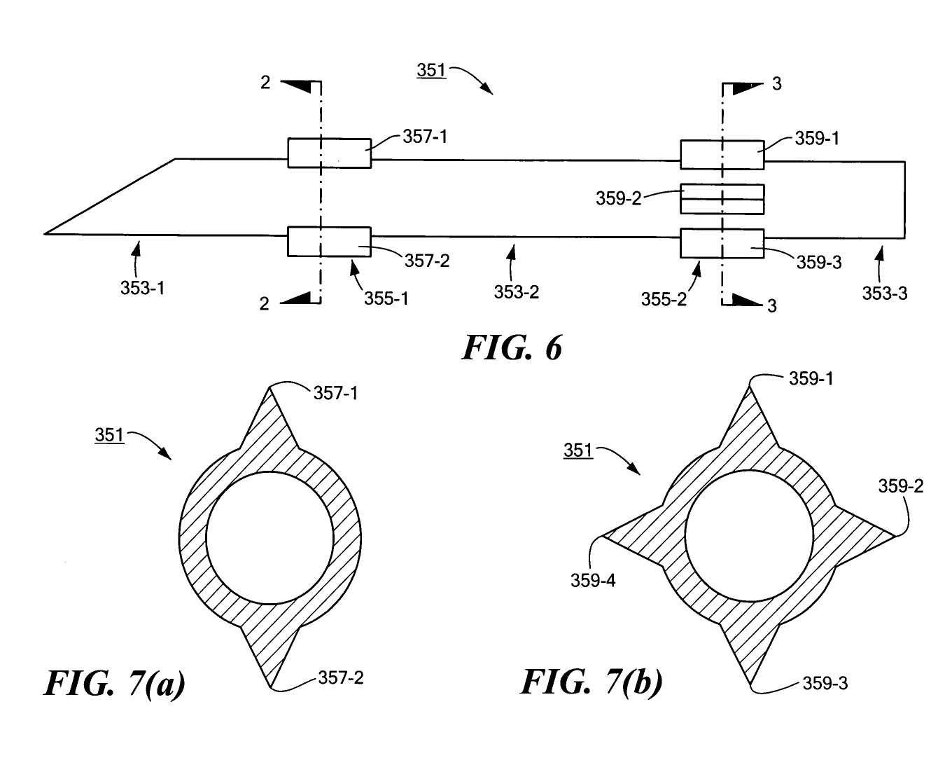

It should also be understood that, although each of the above-described needles has a non-circular cross-sectional shape over its entire length, the present invention is not limited to a needle having a non-circular cross-sectional shape over its entire length, but rather, encompasses a needle having a non-circular cross-sectional shape over only one or more portions of its length. For example, referring now to FIG. 6, there is shown a side view of a further alternate embodiment of a needle for use in echogenic needle aspiration device 11, said needle being represented generally by reference numeral 351. Needle 351 may include alternating lengths of circular cross-sectional shape 353-1 through 353-3 and non-circular cross-sectional shape 355-1 and 355-2. In the present embodiment, lengths 353-1 through 353-3 have substantially the same circular cross-sectional shape as one another; however, it should be understood that lengths 353-1 through 353-3 may alternatively have different circular cross-sectional shapes from one another. As seen best in FIGS. 7(a) and 7(b), lengths 355-1 and 355-2 have different non-circular, cross-sectional shapes from one another, due primarily to the presence of two longitudinally-extending ribs 357-1 and 357-2 in length 355-1 and four longitudinally-extending ribs 359-1 through 359-4 in length 355-2. Because of their different cross-sectional shapes, lengths 355-1 and 355-2 may be used as markings to identify different portions of needle 351.

As can be appreciated, the echogenic needle described above is not limited to use in needle aspiration devices, but rather, may be used in other devices that use hollow needles, such as injection needle devices, or in any other device in which visibility to ultrasound may be desirable.

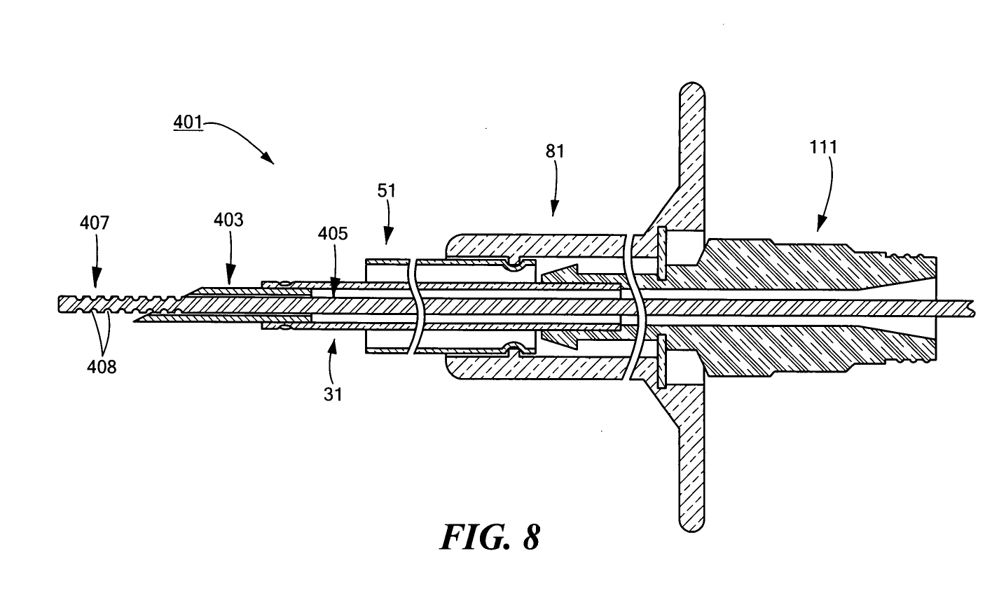

Referring now to FIG. 8, there is shown a fragmentary, longitudinal section view of a second embodiment of an echogenic needle aspiration device constructed according to the teachings of the present invention, the echogenic needle aspiration device being represented generally by reference numeral 401.

Device 401 is similar in most respects to device 11, the principal differences between the two devices being that device 401 may include a hollow needle 403, instead of hollow needle 13, and may additionally include a stylet 405. Needle 403 may have a smooth exterior surface or may have a roughened or echogenic exterior surface that may include one or more voids, grooves, ribs and/or protuberances of the types described in the present specification or described in the patents and publications incorporated by reference into the present specification. In addition, although not shown, needle 403 may alternatively or additionally have a roughened or echogenic interior surface that may include one or more voids, grooves, ribs and/or protuberances of the types described in the present specification or described in the patents and publications incorporated by reference into the present specification.

Stylet 405 has a distal end 407 that may be slidably and removably inserted through the lumen of needle 403. When distal end 407 of stylet 405 is extended distally beyond the distal end of needle 403, stylet 405 may be used to keep matter from entering the lumen of needle 403. At least distal end 407 of stylet 405 may have an echogenic exterior surface that may include one or more echogenic features 408, such as voids, grooves, ribs and/or protuberances of the types described in the present specification or described in the patents and publications incorporated by reference into the present specification. In addition, at least a portion of stylet 405 may include a hardened material to enhance its echogenicity. In this manner, stylet 405 may enhance visualizing when needle 403 has entered a targeted tissue.

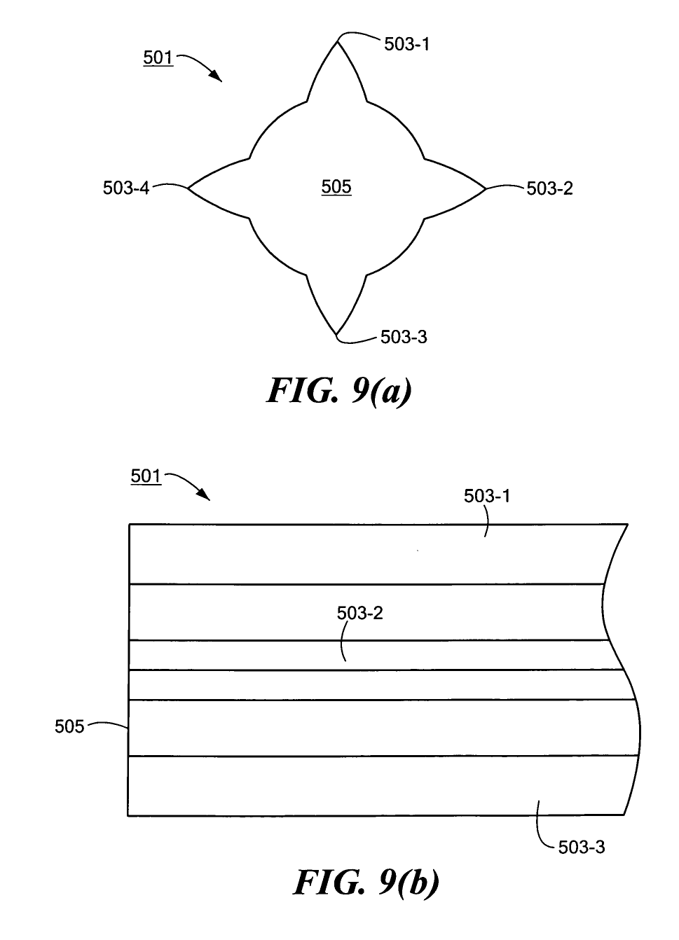

Referring now to FIGS. 9(a) and 9(b), there are shown distal end and fragmentary side views, respectively, of an alternate stylet for use in device 401, said stylet being represented generally by reference numeral 501.

Stylet 501 is similar in many respects to stylet 405, the principal difference between the two stylets being that stylet 501 includes a plurality of longitudinally-extending ribs 503-1 through 503-4 that extend proximally a short distance from distal end 505, ribs 503-1 through 503-4 being spaced around the periphery of distal end 505.

In another embodiment (not shown), the surface of the needle could also be drilled with multiple micro holes and coated with a polymer to modify refraction energy. Alternatively, a bead-blasted surface could be modified to have an impregnated material that reflects the signal differently, such as tungsten or ceramic. Also, the needle surface could be roughened to have a surface like a cheese grater. This type of surface could cut or capture more cells. The grater edges would scrape and having a bigger surface to have the cell protected as it is being retrieved.

In still another embodiment (not shown), a component, which may have little functional strength, may be fit over or inside the needle or over the stylet for enhancing the reflectivity of the device.

The embodiments of the present invention described above are intended to be merely exemplary and those skilled in the art shall be able to make numerous variations and modifications to it without departing from the spirit of the present invention. All such variations and modifications are intended to be within the scope of the present invention as defined in the appended claims.

What is claimed is:

1. An echogenic needle aspiration device comprising:
(a) a first catheter, the first catheter having a proximal end, a distal end and a lumen, the lumen of the first flexible catheter being adapted to receive suction; and
(b) a needle, the needle comprising a proximal end, a distal end and a tubular sidewall, the tubular sidewall defining a lumen and having a length, the proximal end of the needle being fixedly coupled to the distal end of the first catheter, with the lumina of the first flexible catheter and the needle in fluid communication with one another, the tubular sidewall comprising a non-circular transverse cross-section over at least a portion of the length of the tubular sidewall.

2. The echogenic needle aspiration device as claimed in claim 1 further comprising a second catheter, the second catheter having a proximal end, a distal end and a lumen, the needle being slidably insertable into the lumen of the second catheter.

3. The echogenic needle aspiration device as claimed in claim 2 further comprising means for fluidly interconnecting the lumen of the first catheter and a fluid source.

4. The echogenic needle aspiration device as claimed in claim 1 wherein the tubular sidewall comprises a non-circular transverse cross-section over substantially the entirety of the length of the tubular sidewall.

5. The echogenic needle aspiration device as claimed in claim 1 wherein the tubular sidewall includes a plurality of spaced-apart portions of non-circular transverse cross-section.

6. The echogenic needle aspiration device as claimed in claim 5 wherein the plurality of spaced-apart portions of non-circular transverse cross-section differ from one another.

7. The echogenic needle aspiration device as claimed in claim 1 wherein at least a portion of the length of the tubular sidewall has a polygonal shape.

8. The echogenic needle aspiration device as claimed in claim 7 wherein at least a portion of the lumen has a polygonal shape.

9. The echogenic needle aspiration device as claimed in claim 7 wherein at least a portion of the lumen has a circular shape.

10. The echogenic needle aspiration device as claimed in claim 1 wherein at least a portion of the tubular sidewall includes at least one longitudinally-extending projection.

11. The echogenic needle aspiration device as claimed in claim 10 wherein the tubular sidewall includes at least one longitudinal rib.

12. An echogenic medical device comprising:
(a) a needle, the needle comprising a proximal end, a distal end and a lumen; and
(b) a stylet, the stylet being slidably received in the lumen of the needle, the stylet comprising an echogenic distal end.

13. The echogenic medical device as claimed in claim 12 wherein the echogenic distal end of the stylet is shaped to include one or more depressions.

14. The echogenic medical device as claimed in claim 12 wherein the echogenic distal end of the stylet is shaped to include one or more protuberances.

15. The echogenic medical device as claimed in claim 12 wherein the echogenic distal end of the stylet includes a length having a non-circular transverse cross-section.

16. The echogenic medical device as claimed in claim 12 wherein the echogenic distal end of the stylet comprises a composite of structures joined together.

17. A needle visible to ultrasound, the needle comprising a tubular sidewall, the tubular sidewall defining a lumen and having a length, the tubular sidewall comprising a non-circular transverse cross-section over at least a portion of the length of the tubular sidewall.

18. The needle as claimed in claim 17 wherein the tubular sidewall has a non-circular transverse cross-section over substantially the entirety of the length of the tubular sidewall.

19. The needle as claimed in claim 17 wherein the tubular sidewall includes a plurality of spaced-apart portions of non-circular transverse cross-section.

20. The needle as claimed in claim 19 wherein the plurality of spaced-apart portions of non-circular transverse cross-section differ from one another.

21. The needle as claimed in claim 17 wherein at least a portion of the length of the tubular sidewall has a polygonal shape.

22. The needle as claimed in claim 21 wherein at least a portion of the lumen has a polygonal shape.

23. The needle as claimed in claim 21 wherein at least a portion of the lumen has a circular shape.

24. The needle as claimed in claim 17 wherein at least a portion of the tubular sidewall includes at least one longitudinally-extending projection.

25. The needle as claimed in claim 24 wherein the tubular sidewall includes at least one longitudinal rib.

26. The needle as claimed in claim 17 wherein the tubular sidewall includes a hardened portion.
